(19) United States
(12) Patent Application Publication
(10) Pub. No.: US 2008/0319258 A1
(43) Pub. Date: Dec. 25, 2008

(54) Sterile Access Conduit

(76) Inventor: Christopher C. Thompson, Needham, MA (US)

Correspondence Address: FISH & RICHARDSON PC, P.O. BOX 1022, MINNEAPOLIS, MN, 55440-1022, US

(21) Appl. No.: 11/994,678

(22) PCT Filed: Jul. 14, 2006
(86) PCT No.: PCT/US2006/027299

§ 371 Date: Aug. 12, 2008

Related U.S. Application Data
(60) Provisional application No. 60/699,785, filed on Jul. 15, 2005.

(51) Int. Cl. A61B 1/018

(52) U.S. Cl. 600/104

(57) ABSTRACT

An elongate flexible conduit including a sealing membrane is positioned through a natural body orifice and along the gastrointestinal (GI) tract of a patient. The sealing membrane is advanced along the GI tract until proximate a target wall segment of an inner wall of the GI tract. The sealing membrane is then permanently or releasably attached to the target wall segment thereby sealing off and isolating the target wall segment from the rest of the GI tract establishing a sterile pathway from the target wall to the natural body orifice. An incision is made through both the sealing membrane and the wall of the GI tract to gain access to the peritoneum to perform surgical techniques without allowing bacteria, stomach acids, and other contaminants in the GI tract to enter the peritoneum. An instrument can then be introduced into the peritoneal cavity through the incision along the sterile pathway.

### TECHNICAL FIELD

This invention relates generally to endoscopic surgery, and more particularly to natural orifice (per-oral and per-anal) transluminal endoscopic surgery.

### BACKGROUND

Standard surgical approaches for abdominal surgery include minimally invasive surgical procedures such as laparoscopic and endoscopic procedures, for example, through small incisions in the abdominal wall. Such surgery requires general anesthesia with its inherent risks and complications, post-operative abdominal wall pain, prolonged hospital stays, and may be difficult in obese patients. Resulting abdominal scars are unaesthetic and can potentially result in complications such as wound infection, rupture, and development of adhesions and hernias. Minimally invasive surgical techniques can only minimize these drawbacks.

### SUMMARY

The invention is based, at least in part, on the discovery that if one can securely engage a flexible sealing membrane to a target segment of an inner wall of the gastrointestinal (GI) tract, thereby sealing off and isolating the target wall segment from the rest of the GI tract, one can cut through both the sealing membrane and the wall of the GI tract to gain access to a patient's peritoneum, mediastinum or other body cavity to perform surgical techniques without allowing bacteria, stomach acids, and other contaminants in the GI tract to enter the peritoneum.

In one aspect, the invention features methods of endoscopically accessing the peritoneal cavity of a patient by positioning an elongate flexible conduit including a sealing membrane through a natural body orifice and along a gastrointestinal tract of a patient; advancing the sealing membrane along the gastrointestinal tract until proximate a target wall segment of the gastrointestinal tract; attaching the sealing membrane to the target wall segment; forming an incision in the sealing membrane and target wall segment attached thereto; and introducing an instrument through the incision and into the peritoneal cavity.

In certain embodiments, one can permanently or releasably attach the sealing membrane to the target wall segment. In various procedures, the natural body orifice is the mouth of the patient, the anus of the patient, or a plurality of flexible conduits are positioned along the gastrointestinal tract substantially simultaneously. As part of the above described aspect, one can also detach the sealing membrane from the flexible conduit. The sealing membrane can be detached from the flexible conduit by a trigger component. One can also position an overtube through the mouth of the patient along the esophageal passage as part of the above procedure.

In another aspect, the invention features an endoscopic access device including an elongate tube defining an interior passage sized to receive an instrument; a sealing membrane positioned at the distal end of the tube, wherein the sealing membrane is positionable in a stowed state and a deployed state; and a selectively activatable adhesive disposed on an outer side of the sealing membrane for engagement with a wall segment of the gastrointestinal tract.

In some embodiments, the sealing membrane further includes a deployable cuff positionable in a stowed state and a deployed state. The cuff can be sized for passage through a natural body orifice in the stowed state and the cuff is sized to securely engage a target inner wall segment of the gastrointestinal tract in the deployed state.

In another aspect, the invention features an endoscopic access device including an elongate tube defining an interior passage sized to receive an instrument and a deployable cuff positionable in a stowed state and a deployed state. The cuff is sized for passage through a natural body orifice in the stowed state and the cuff is sized to securely engage a target inner wall segment of gastrointestinal tract in the deployed state.

In various embodiments, at least a portion of the sealing membrane is substantially transparent. The adhesive can be activated by a predetermined temperature, a predetermined pH level, exposure to ultraviolet light, exposure to a chemical agent, the application of pressure and/or exposure to a body fluid. The adhesive can also be remotely activatable. The adhesive can include a biocidal and/or antibacterial gel or such gels can be applied separately to the sealing membrane. The adhesive can be substantially permanent or substantially biodegradable. The adhesive can also be configured to degrade over a predetermined time. In some embodiments, the sealing membrane is substantially biodegradable.

In various embodiments, the adhesive is configured to deactivate upon application of a predetermined temperature, pH level, a predetermined moisture level and/or a chemical agent.

In some embodiments, the device includes a frangible zone located between the elongate tube and the sealing membrane. The frangible zone can be configured to tear and separate the sealing membrane from the elongate tube upon application of a predetermined force.

In some embodiments, the device includes a trigger component located near the sealing membrane and configured to release a distal end of the elongate tube from the sealing membrane when actuated. The trigger component can be located near the sealing membrane and can be configured to release the sealing membrane from the wall segment of the gastrointestinal tract when actuated.

In some embodiments, the access device includes an actuator located at a proximal end of the elongate tube and operatively connected to the trigger component. The actuator can be configured to release the distal end of the flexible tube from the sealing membrane when actuated.

In further embodiments, the access device includes an actuator located at a proximal end of the elongate tube and operatively connected to the trigger, the actuator can be configured to release the sealing membrane from the wall segment of the gastrointestinal tract when actuated.

In another aspect, the invention features methods of endoscopically accessing the abdominal cavity of a patient by positioning an access device through a natural body orifice and along the gastrointestinal tract of a patient; advancing the sealing membrane along the gastrointestinal tract until proximate a target wall segment of the gastrointestinal tract; activating an adhesive disposed on an outer side of the sealing membrane; attaching, e.g., releasably attaching, the sealing membrane to the target wall segment; and forming an incision in sealing membrane and target wall segment attached thereto to provide access to the peritoneal cavity.

In another aspect, the invention features method of endoscopically accessing the peritoneal cavity of a patient by positioning an access device through a natural body orifice and along the gastrointestinal tract of a patient; advancing the cuff along the gastrointestinal tract until proximate a target wall segment of the gastrointestinal tract; changing the cuff from a stowed to a deployed state to releasably secure the cuff to the target wall segment; and forming an incision in the cuff and target wall segment secured thereto to provide access to the peritoneal cavity.

A patient can be a human or an animal, e.g., a mammal, or any domesticated or wild animal having a gastrointestinal tract, such as a dog, cat, horse, pig, cow, goat, ape, or monkey.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the description, and from the claims.

### DESCRIPTION OF DRAWINGS

FIG. 1 is a schematic representation showing a plan view of an embodiment of the access device including a sealing membrane in a stowed state.

FIG. 2A is a schematic representation showing a plan view of the access device of FIG. 1 wherein the sealing membrane is in a deployed state.

FIG. 2B is a schematic perspective representation of the access device of FIG. 2A.

FIG. 3 is a schematic representation showing a plan view of another embodiment of the access device including a sealing membrane in a stowed sate.

FIG. 4A is a schematic representation showing a plan view of another embodiment of the access device including a sealing membrane in a stowed state.

FIG. 4B is a schematic representation showing a plan view of another embodiment of the access device including a sealing membrane in a deployed state.

FIG. 4C is a schematic perspective representation of the access device of FIG. 4B.

FIG. 5A is a schematic cut-away view of a stomach showing the device of FIG. 4B with a sealing membrane in a stowed state and located proximate a target wall segment of the stomach via the esophagus.

FIG. 5B is a schematic cut-away view of a stomach showing the device of FIG. 4B with a sealing membrane in a deployed state and located proximate a target wall segment of the stomach via the esophagus.

FIG. 5C is a schematic cut-away view of a stomach showing the device of FIG. 4B with a sealing membrane in a deployed state with an incision made in the sealing membrane and target wall segment of the stomach.

FIG. 6A is a schematic representation showing a plan view of another embodiment of the access device including an elastic cuff in a stowed state.

FIG. 6B is a schematic representation showing a plan view of the access device of FIG. 6A in a deployed state.

FIG. 7A is a schematic representation showing a plan view of another embodiment of the access device including an elastic cuff and a flexible distal tip to engage an endoscope (in a stowed state).

FIG. 7B is a schematic representation showing a plan view of the access device of FIG. 7A in a deployed state.

FIG. 8A is a schematic representation showing a partial cut-away view of the colon with the device of FIG. 1 or 2 including a sealing membrane positioned in a stowed state located proximate a target wall segment of the colon via the anus.

FIG. 8B is a schematic representation showing a partial cut-away view of the colon with the sealing membrane positioned in a deployed state located proximate a target wall segment of the colon via the anus.

FIG. 8C is a schematic representation showing a partial cut-away view of the colon with the sealing membrane positioned in a deployed state with an incision made in the sealing membrane and target wall segment of the colon.

FIG. 9A is a schematic representation showing a partial cut-away view of the colon with the device of FIGS. 7A and 7B including a sealing membrane as part of an elastic cuff positioned in a stowed state located proximate a target wall segment of the colon via the anus.

FIG. 9B is a schematic representation showing a partial cut-away view of the colon with the sealing membrane of FIG. 9A positioned in a deployed state located proximate a target wall segment of the colon via the anus.

FIG. 9C is a schematic representation showing a partial cut-away view of the colon with the sealing membrane of FIGS. 9A and 9B positioned in a deployed state located proximate a target wall of the colon with an incision made in the sealing membrane and target wall of the colon.

FIGS. 10A and 10B show schematic cross-sectional views of a sterile overtube.

Like reference symbols in the various drawings indicate like elements.

### DETAILED DESCRIPTION

Traditionally, natural orifice (per-oral/per-anal) endoscopy has been limited to techniques performed within the intestinal lumen, such as diagnostic colonoscopy, diagnostic upperendoscopy, polyp removal, and treatment of gastrointestinal bleeding. In one aspect, the present invention provides methods, systems, and devices for performing natural-orifice transluminal surgery. Specifically, a flexible endoscope is advanced along a sterile pathway established from the mouth or anus and through an incision made in the wall of the stomach, small bowel, or colon to perform abdominal surgery. By avoiding incisions through the abdominal wall, this approach may reduce post-operative complications, including: abdominal wall pain, wound infections, herniations, rupture, and adhesions. The transluminal surgical methods described herein are also advantageous in the morbidly obese and those too ill to undergo traditional surgery. A variety of procedures can be performed using these new methods including: abdominal exploration, cholecystectomy, spleenectomy, hepatic resection, oopherectomy, hysterectomy, tubal ligation, and various thoracic and weight loss procedures.

### General Methodology

A per-oral approach was developed as a minimally invasive alternative to current abdominal and pelvic surgical procedures. The technique involves placing an endoscope through the mouth and into the stomach and then puncturing the stomach to gain access to the peritoneal cavity. The endoscope is then advanced into the peritonial cavity where a surgical procedure is performed. This procedure does not require any abdominal incisions. Potential benefits of such a procedure include fewer problems with wound complications such as infections and herniations, which may be particularly important in the obese population. The less invasive nature allows for a quicker recovery, which may be important from economic and quality of life stand points and also likely to have a large impact in those who are terminally ill with a short life expectancy. Other potential benefits include fewer problems with intra abdominal adhesions and lower anesthesia and sedation requirements.

Examples of procedures where this approach may be preferable to an open or laparoscopic approach include: (1) procedures that are not technically demanding such as abdominal exploration, peritonial biopsy, exploration and liver biopsy, and lyses of adhesions; (2) palliative procedures in the terminally ill, including gastric bypass in patients with pancreatic, biliary, or duodenal cancers; (3) bariatric procedures such as Roux-n-y gastric bypass. This minimally invasive approach can also be adapted to other intra-abdominal procedures such as cholecystectomy, appendectomy, splenectomy, partial hepatic resection, colorectal surgery, oophorectomy, and hysterectomy.

The per-anal (trans-anal) and/or trans-colonic approaches provide additional benefits compared to the per-oral approach as they enable straight access to most upper abdominal organs, however, with either approach (per-oral or per-anal) there is a significant risk of dangerous contamination from oral or colonic flora. Accordingly, such procedures should be done only with the use of a new so-called “sterile conduit” described herein, which enables a sterile and safe access to the peritoneal cavity. Of course, there are also benefits of using the sterile conduit for per-oral techniques. For example, the sterile conduit will prevent any contents in the stomach, such as acids, from contaminating the peritoneal cavity.

In some embodiments, the conduit is configured to be advanced and positioned on a target segment of bowel wall, reversibly attached to the bowel wall, and then enable passage of a standard therapeutic endoscope. In certain embodiments, the device is configured to attach to the wall of the esophagus, stomach, small bowel, or colon prior to accessing the peritoneal cavity to isolate the target wall segment from the rest of the GI tract and to assure sterility. In some embodiments, the distal end can be sized and configured to permit organ resection. In some embodiments, the distal end of the device is configured to be substantially air tight to allow pneumoperitoneum and intra-abdominal pressure monitoring. It is further contemplated that the techniques disclosed herein may be used in lieu of, or in connection with, open or laparoscopic surgical methods where tissue manipulation and fixation are required.

Sterile Conduits with Sealing Membranes

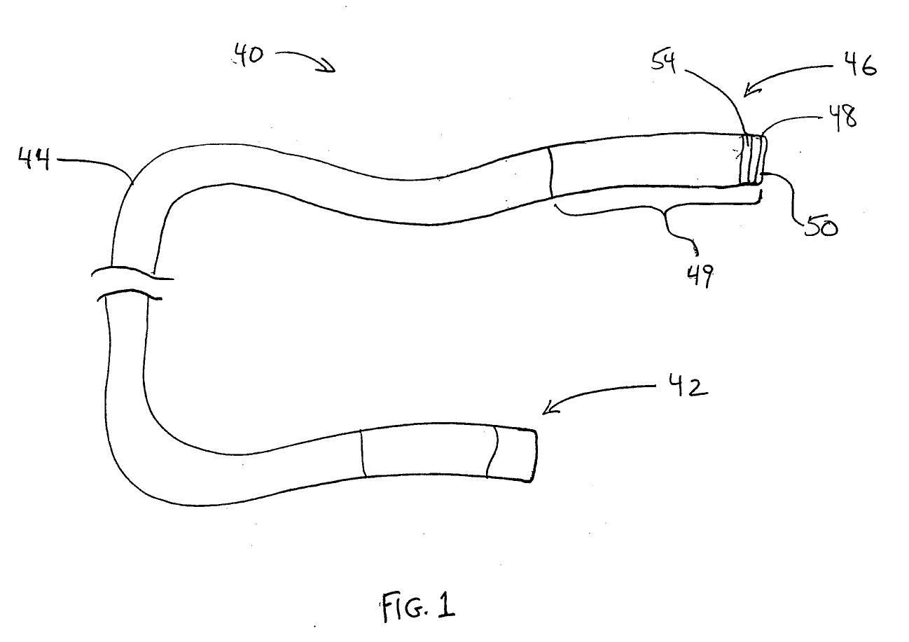

FIG. 1 shows an access device 40 according to one embodiment, and includes an open proximal end 42, an elongate body 44, and a closed distal end 46. In some embodiments, the access device 40 is formed of a compliant material. In some embodiments the access device 40 is formed of a flexible material, and is sized with a large enough diameter to allow tissue or organ extraction though it, and/or device placement via the conduit.

A sealing membrane 48 is formed on the distal end 46 of a distal segment 49. The sealing membrane 48 can be coated partially or completely with an adhesive layer 50. In some embodiments, at least a portion of the device 40, and at least the sealing membrane 48, is substantially transparent to allow the surgeon to visualize the target wall segment to make the initial cut through the GI tract wall. The sealing membrane must be sufficiently flexible to follow the contours of the target wall segment, yet be sufficiently strong to form a secure seal against the wall. The sealing membranes can be made of various know elastomeric materials, such as including but not limited to cyanoacrylates, fibrin, collagen, and hydrogel sealants. The membranes can also be formed of materials such as elastomeric urethanes (TPU), polyamid elastomerics (PEBAX®), polysester elastomerics (HYTREL®), styrene block copolymers (KRATON®), natural rubbers and/or vulcanized polyisoprene.

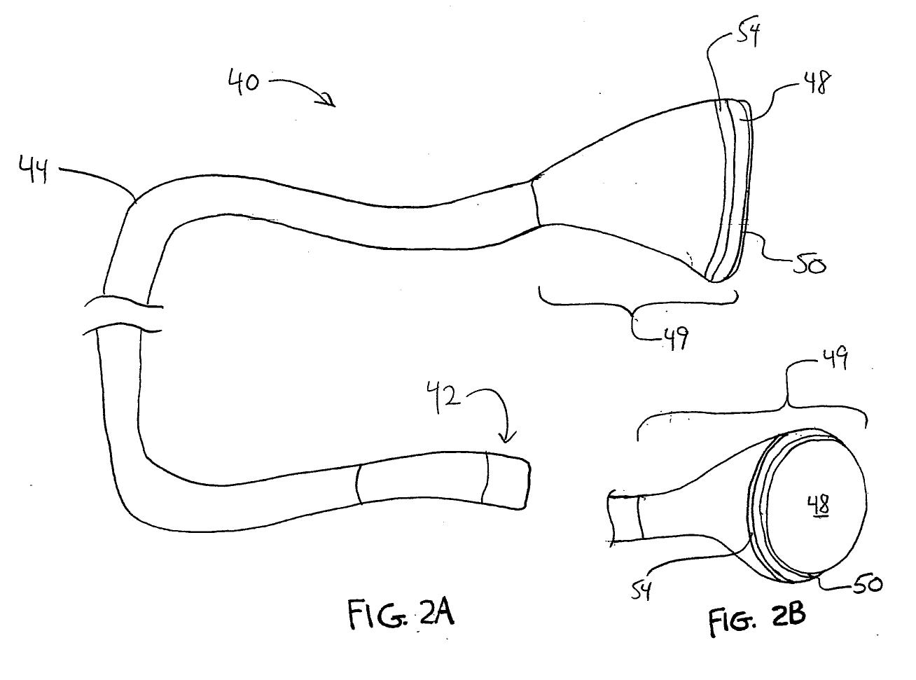

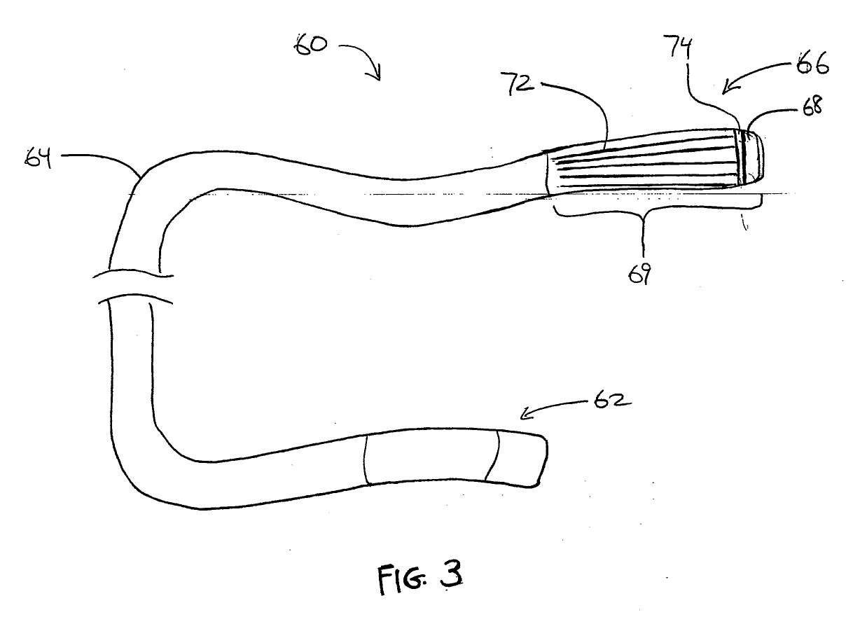

The adhesive layer should be biocompatible, and can be a releasable or substantially permanent adhesive. The sealing membrane 48 can be integrally formed with the elongate body 44 or attached thereto by ultrasonic welding, for example. In some embodiments, the wall thickness of the sealing membrane 48 is sufficiently thin relative to the wall thickness of the elongate body 44 such that delivering a pressurized gas to the device 40 from the proximal end 42 preferentially expands the sealing membrane 48 with substantial lateral expansion of the elongate body 44 to form a cone shape as shown in FIG. 2. Other three-dimensional shapes such as hemispheres, pyramids, cylinders, and prisms, can also be used. Accordingly, the sealing membrane can be positioned in a closed or stowed state (FIG. 1) for passage through a body lumen and an expanded, unfurled, or deployed state (FIGS. 2A and 2B). The device 40 can also include a frangible region 54 disposed between the sealing membrane 48 and the remaining portion of the distal segment 49, having a weakened tensile strength relative to the elongate body 44 and configured to tear upon application of a predetermined force.

As shown in FIG. 3, an access device 60 according to another embodiment, includes an open proximal end 62, an elongate body 64, and a closed distal end 66. A sealing membrane 68 is formed on the distal end 66 of a distal segment 69. The sealing membrane 68 can be coated partially or completely with an adhesive layer 70. The distal segment 69 can include one or more pleats or folds 72 to permit expansion of the distal segment 69 when a pressurized gas is delivered to the device 60 from the proximal end 62. The device 60 can also include a frangible region 74 disposed between the sealing membrane 68 and the remaining portion of the distal segment 69.

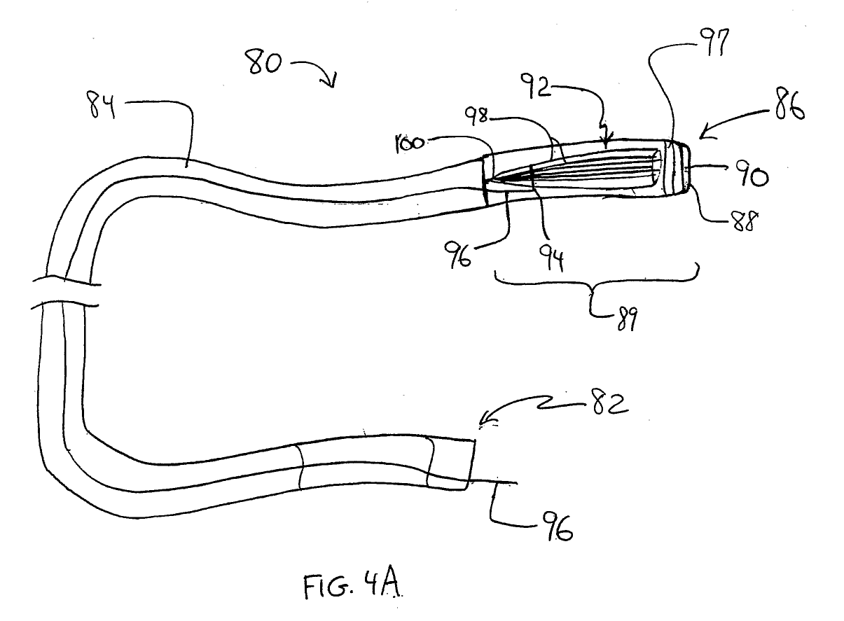

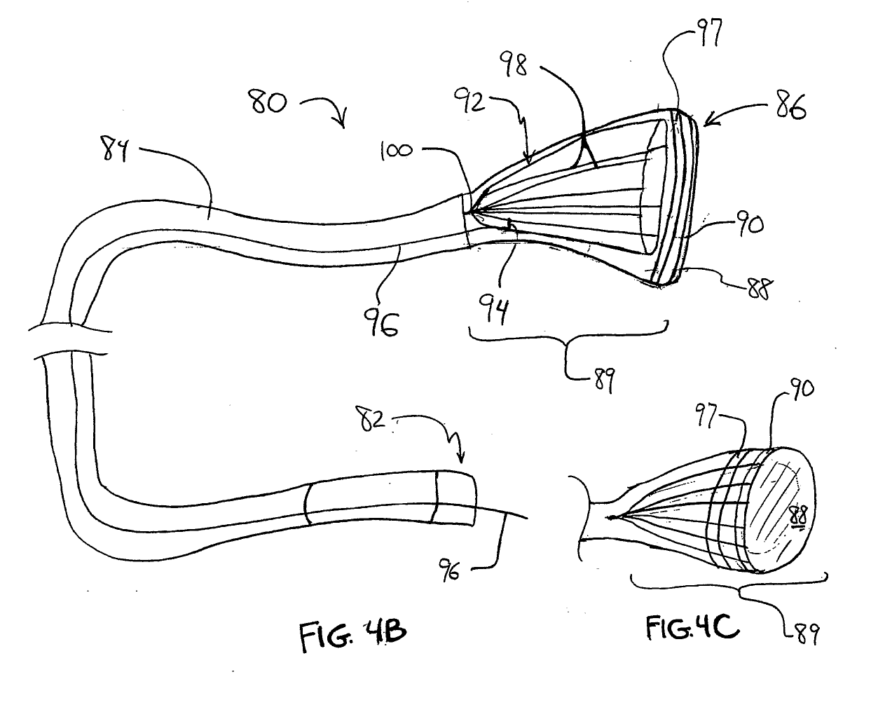

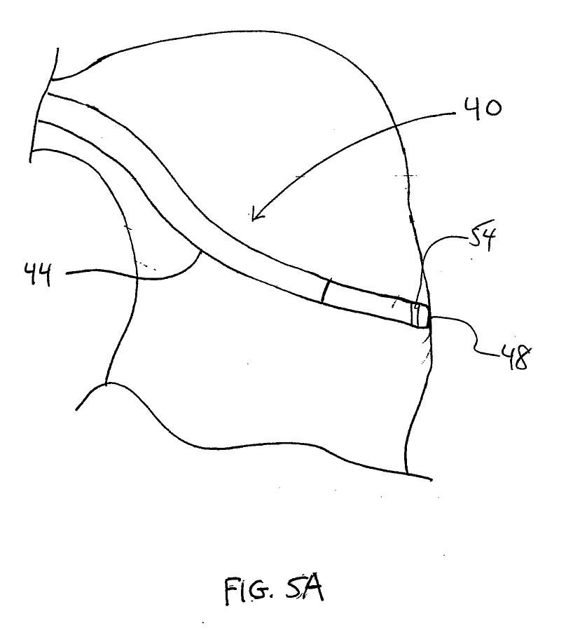

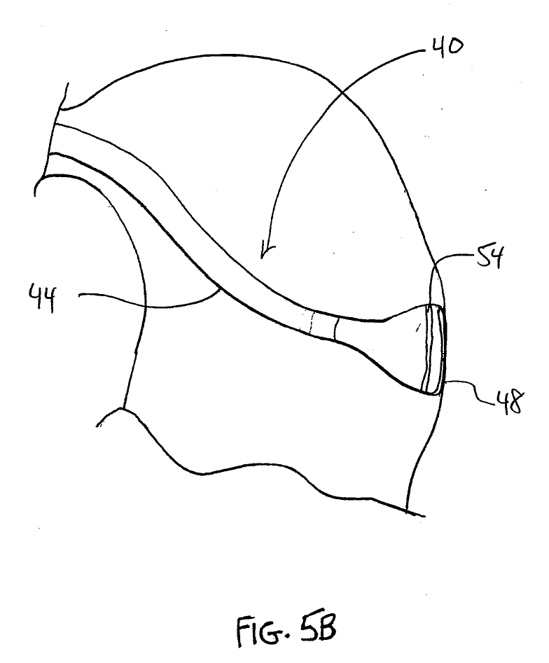

FIG. 4A shows an access device 80 according to another embodiment, which includes an open proximal end 82, an elongate body 84, and a closed distal end 86. A sealing membrane 88 is formed on the distal end 86 of a distal segment 89. The sealing membrane 88 can be coated partially or completely with an adhesive layer 90. The sealing membrane 88 includes a deployment assembly 92, a trigger 94, and an actuator 96, which are operably connected to the deployment assembly 92. The actuator 96 can include a suture attached to the trigger 94 and extending through the elongate body 84 and out of the proximal end 82. In one embodiment, the deployment assembly 92 includes a plurality of arms 98 rotatably connected at coupler 100 and biased radially outward. The arms can be made from any of several alloys of nickel and titanium that return to their original shape after deformation, such as nitinol, for example. The trigger 94 binds the arms together and maintains the sealing membrane 88 of the device in the stowed state. In one embodiment, the trigger 94 is released by applying sufficient tension to the actuator 96, thereby permitting the arms 98 to move radially outwardly and position the sealing membrane 88 to the deployed state as shown in FIGS. 4B and 4C. The device 80 can also include a frangible region 97 disposed between the sealing membrane 88 and the remaining portion of the distal segment 89.

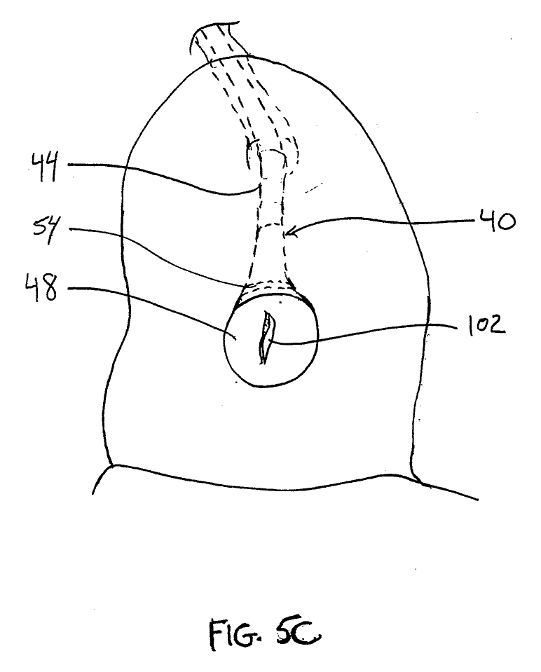

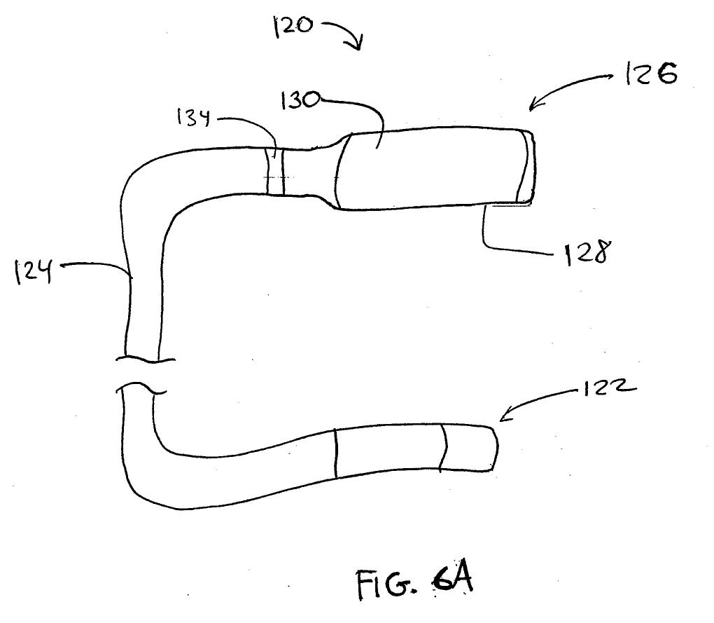

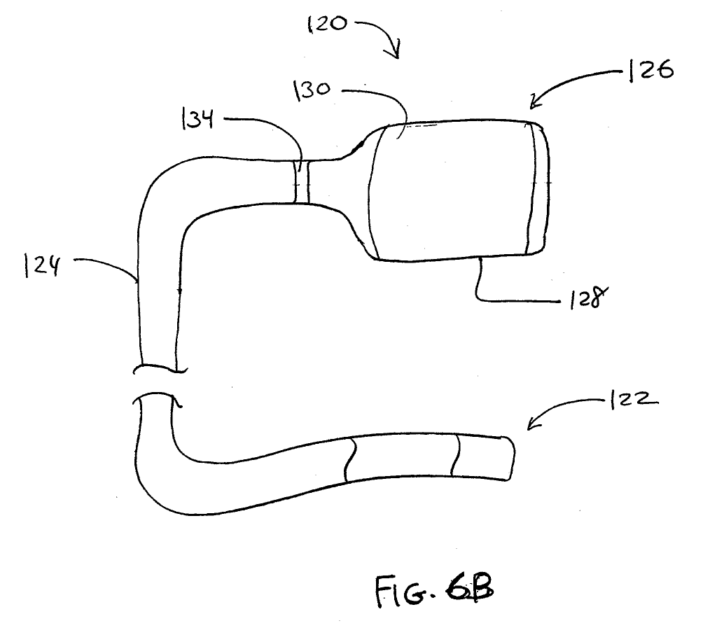

FIG. 5A is a schematic cut-away view of a stomach showing the access device 40 with a sealing membrane 48 in a stowed state and located proximate a target wall of the stomach via the esophagus. As shown in FIG. 5B, the sealing membrane 48 is then changed to the deployed state and at least a portion of the sealing membrane is attached to the target wall segment of the stomach with an adhesive. Examples of such adhesives include cyanoacrylates, fibrin, and hydrogel sealants. The foregoing can be chemically cross-linked at the target wall segment, for example. In some embodiments, the adhesive applied to the access device 40 is activated at a first predetermined temperature, such as internal body temperature. The adhesive can be deactivated at a second predetermined temperature. In some embodiments, a cool or warm saline solution can be delivered toward the adhesive through the device 40 to reach the first or second predetermined temperatures. In other embodiments, the adhesive is activated at a predetermined pH level, exposure to electromagnetic radiation, e.g., ultraviolet or visible light, or exposure to a body fluid.

In other embodiments, the adhesive is remotely activatable. For example, a UV curable adhesive can be applied to the substantially transparent sealing membrane 48. One such adhesive is described in U.S. Pat. No. 5,948,427, having an issue date of Sep. 7, 1999. Once the sealing membrane 48 is positioned to the target wall of the stomach (FIG. 5B), light from a tunable nitrogen dye laser, for example, adjusted to the appropriate UV range, is transmitted down a cannula (not shown), which is advanced along the elongate body 44, and is then transmitted at 360 degrees within the distal end 46 of the device. Thus, the UV light passes through the UV-transparent plastic sealing membrane 48, and activates or cures the adhesive, while simultaneously sterilizing the field between the sealing membrane and the target wall segment. Other UV light sources include a xenon lamp in combination with fiber optic cables, fiber optic retractors, and instruments and/or a four port rotating light cable adapter, for endoscopic procedures.

This UV sterilization provides a sterile, isolated environment that is maintained even after the surgeon cuts through the sealing membrane and the target wall segment to maintain access to the peritoneum. The sealing membrane and adhesive provides a secure seal to ensure that no contaminants from the stomach can pass into the peritoneum.

Examples of photochemically driven surgical tissue adhesives include UV-curable polyester polyol acrylates, photoreactive gelatins and water-soluble difunctional macromers (poly(ethylene glycol) diacrylate; PEGDA). The gelatins are partially derivatized with photoreactive groups, such as UV-reactive benzophenone, or visible light-reactive xanthene dye (e.g., fluorescein sodium salt, eosin Y, and rose bengal). These photocurable tissue adhesive glues are viscous solutions under warming, and irradiation by UV or visible light for 1 minute produces water-swollen gels, which have a high adhesive strength to tissues. An increase in the irradiation time can result in increased gel yield and reduced water swellability. A decrease in the molecular weight of PEGDA and an increase in concentration of both gelatin and PEGDA can result in reduced water swellability and increased tensile and burst strengths of the resultant gels. These glues gradually degrade with time. Nakayama et al., J. Biomed. Mater. Res., 1999; 48(4):511-21.

In other embodiments, the adhesive is activated with pressure or exposure to a chemical agent. The adhesive can include a biocidal and/or antibacterial gel. In certain embodiments, the adhesive is substantially permanent, while in other embodiments the adhesive is selected to biodegrade after several hours, days, or weeks following the surgery. For example, one known biodegradable tissue adhesive is 1,2-isopropylidene glyceryl 2-cyanoacrylate.

FIG. 5C shows the sealing membrane 48 attached to the target wall of the stomach with an incision 102 made in the sealing membrane 48 and the target wall with a needle knife, for example (not shown) advanced along the elongate body 44 of the access device 40. In so doing, a sterile pathway is established to the peritoneal cavity. Transgastric endoscopic procedures can then be performed using a standard therapeutic endoscope as described in U.S. Patent Publication Number US 2001/0049497, for example.

In some embodiments, the access device 40 can include a pressure gauge or pressure monitor (not shown) in communication with the elongate tube 44 to permit monitoring of pneumoperitoneum and intra-abdominal pressure. The access device 40 can also include a pressure controller (not shown) in communication with the elongate tube 44 to selectively control the pressure by introducing gas into, or evacuating gas from the device.

Once the endoscopic procedure is completed, the incision 102 is closed and the sealing membrane 48 is either removed from the target wall segment of the stomach along with the elongate body 44, or the elongate body 44 is separated from the sealing membrane 48 along the frangible region 54 by the application of force to the distal portion 42, leaving the sealing membrane in place when the elongate body is removed. In this latter embodiment, the sealing membrane 48 remains attached to the target wall of the stomach and either remains inert or biodegrades at a known rate.

In other embodiments, the adhesive is substantially biodegradable, and can be specified to degrade over a predetermined time until the sealing membrane 48 is released from the target wall segment of the stomach. Once released, the sealing membrane passes harmlessly through the rest of the GI tract and is passed out of the body. The adhesive can also be specified to deactivate upon application of at least one of a predetermined temperature, pH level, a predetermined moisture level, or a chemical agent.

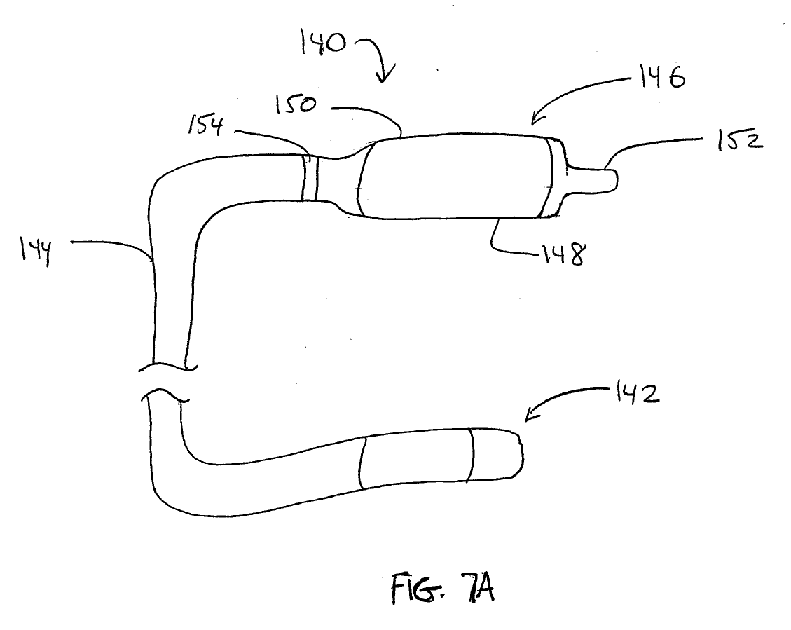

FIG. 6A shows an access device 120 according to another embodiment. This device includes an open proximal end 122, an elongate body 124, and a closed distal end 126. A sealing membrane 128 is arranged on the surface of or as an integral part of a deployable elastic cuff that is formed on the distal end 126. The sealing membrane can be coated partially or completely with an adhesive and/or sealing layer 130. In some embodiments, at least a portion of the device 120, e.g., the sealing membrane 128, is substantially transparent. The adhesive and/or sealing layer can include a biocompatible adhesive for releasable or substantially permanent attachment to a body lumen target wall segment. The sealing membrane 128 is configured for passage along a lumen in the GI tract, e.g., the colon of a patient, while in the stowed state and can be secured to a target wall segment of the colon, or any other tubular portion of the GI tract, in a deployed state as shown in FIG. 6B. In some embodiments, the sealing membrane 128 is also located at the flat distal end 126 of device 120, and is used in a manner similar to the devices shown in FIGS. 2B and 4B when the cuff is expanded. This embodiment provides a dual-purpose device, that can have a sealing membrane around the periphery of an expandable cuff, as well as at an essentially perpendicular flat end of the cuff. This device can be used anywhere in the GI tract.

The wall thickness of the sealing membrane 128 (and, if present, the underlying wall of the elastic cuff) can be sufficiently thin relative to the wall thickness of the elongate body 124 such that delivering a pressurized gas to the device 120 from the proximal end 122 preferentially expands the sealing membrane 128. Alternatively, the sealing membrane can include a toroidal chamber (not shown) which is expanded by pressurized gas delivered through a lumen extending along, e.g., within, the elongate body 144.

The device 120 can also include a frangible region 134 having a weakened tensile strength relative to the elongate body 44 and configured to tear upon application of a predetermined force as described above with respect to access devices 40, 60, and 80.

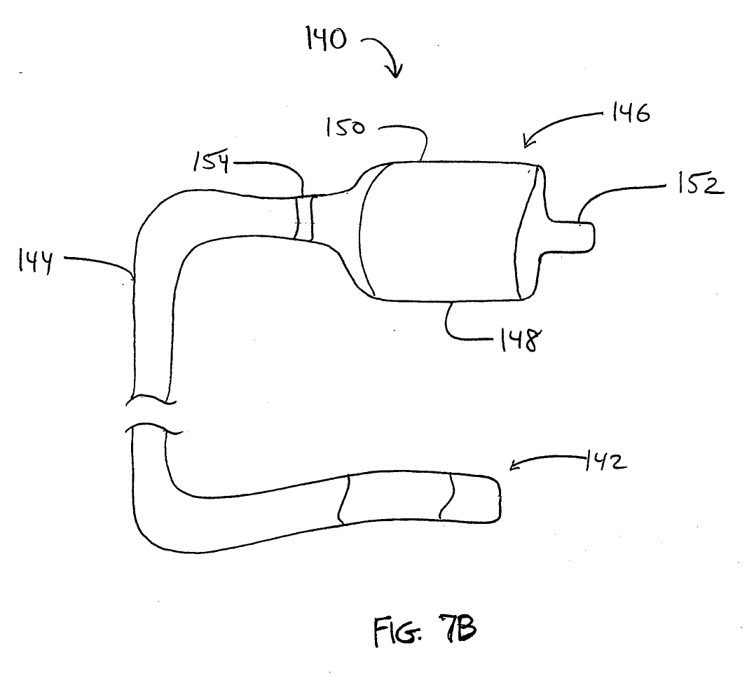

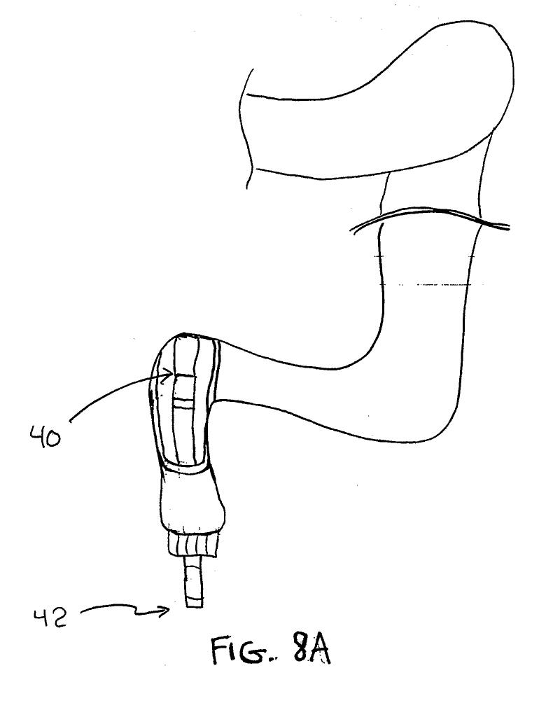

FIG. 7A shows an access device 140 according to another embodiment. This device includes an open proximal end 142, an elongate body 144, and a closed distal end 146. A sealing membrane 148 including (or part of) a deployable elastic, expandable cuff is formed on the distal end 146, which can be coated partially or completely with an adhesive and/or sealing layer 150. The distal end 146 includes a narrowed distal tip 152 for snugly receiving the distal end of a therapeutic endoscope that is used to steer the access device 140. When an endoscope is positioned within the distal tip 152, greater control is possible as the distal end 146 is advanced along the colon of a patient for example.

The device 140 can also include a frangible region 154 having a weakened tensile strength relative to the elongate body 144 that is configured to tear upon application of a predetermined force as described above with respect to access devices 40, 60, 80, and 120.

In some embodiments, at least a portion of the device 140 is substantially transparent. The adhesive and/or sealing layer can include be a biocompatible adhesive for releasable or substantially permanent attached to a body lumen wall. The sealing membrane 148 is configured for passage along the colon of a patient while in the stowed state and can be secured to a target wall of the colon in a deployed state as shown in FIG. 7B.

The adhesive and/or sealing layer can, in this embodiment, have only a sealing function, as the elastic cuff may be held in place by air pressure or other pressure that keeps the cuff in the expanded, deployed position. Thus, no adhesive is required in this embodiment, although in most cases, one may still benefit from the use of a sealing layer that also has adhesive qualities.

In some embodiments, the elastic cuff includes a mechanical framework disposed within the cuff and configured to expand the cuff to secure it in position against the a target wall segment without or in combination with air pressure. The framework can include a very large cell stent, made of plastic, stainless steel or other metal or shape memory alloy configured to spring into a deployed or open position under a specific triggering event (e.g., being pushed out of a sheath within the elongate conduit to open and pulled back into the sheath to close). The open diameter of the cuff would be selected to be larger than the diameter of the GI tract to be isolated, thus creating a pressure fit of the cuff within the tract.

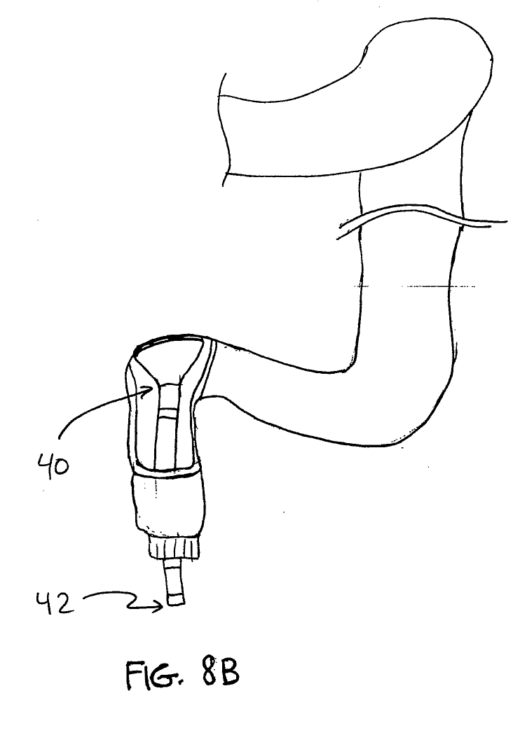

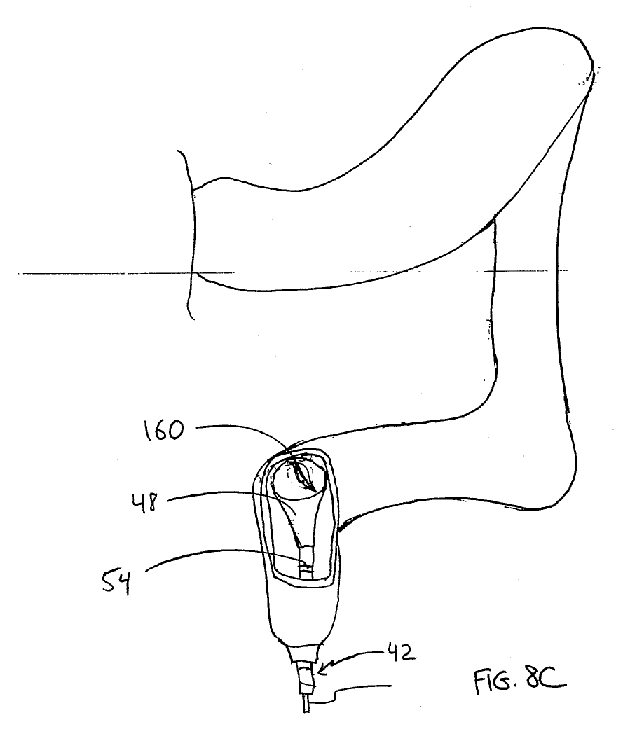

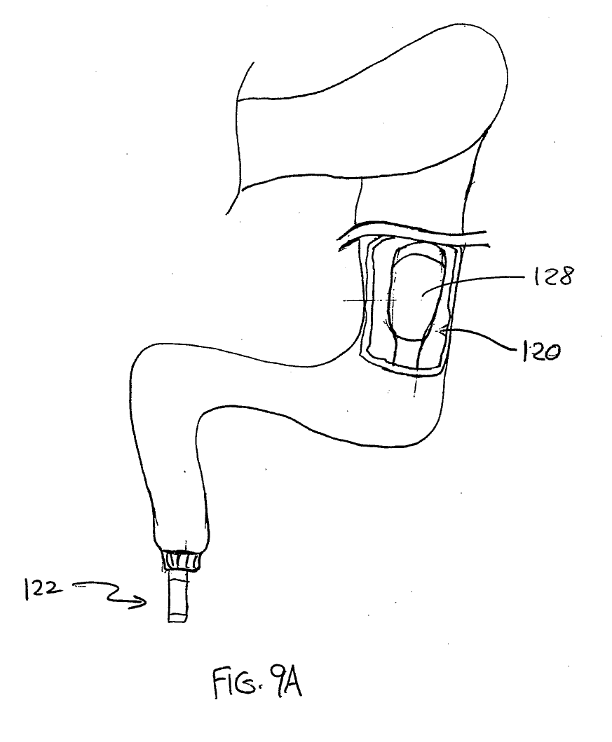

FIGS. 8A to 8C show a partial cut-away view of the colon with the access device 40 with the sealing membrane 48 positioned in a stowed state and located proximate a target wall segment of the colon via the anus. As shown in FIG. 8B, the sealing membrane 48 is then expanded to its deployed state and the sealing membrane 48 is attached to the target wall segment of the colon with an adhesive. The adhesive can have one or more of the properties described herein. When using certain adhesives, such as adhesives containing fibrin or collagen, for example, the mucosa can be ablated, disrupted or agitated before application of the adhesive, using for example, electrosurgery, electrocautery, argon plasma coagulation, mechanical disruption, such as a mucosa stripping device or brush, or any combination of the foregoing.

FIG. 8C shows the sealing membrane 48 attached to the target wall of the colon with an incision 160 made in the sealing membrane 48 and the target wall with a needle knife, for example (not shown) advanced along the elongate body 44 of the access device 40. In so doing, a sterile pathway is established to the peritoneal cavity. In some embodiments, the device 40 can be air tight to allow pneumoperitoneum and intra-abdominal pressure monitoring. The access device can be removed as described above.

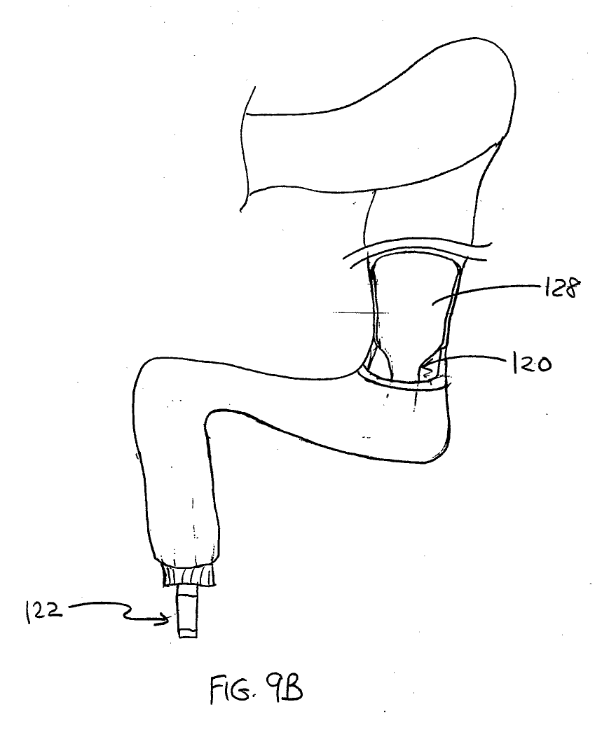

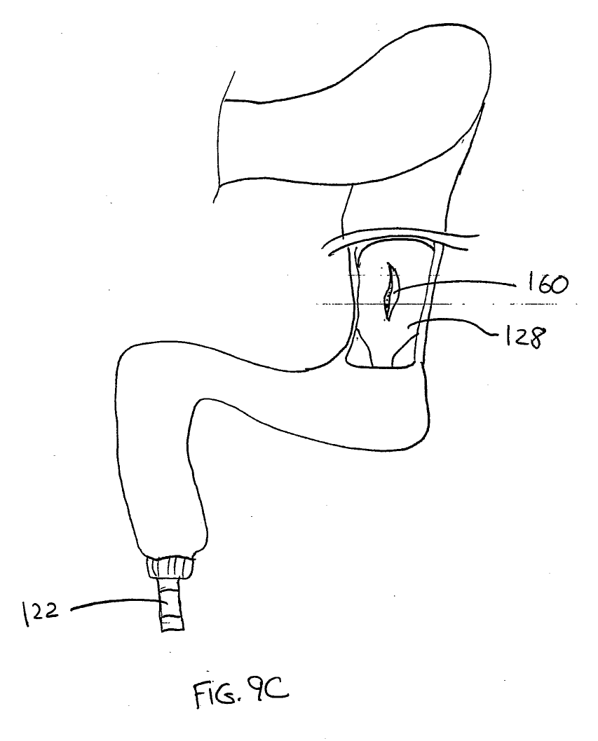

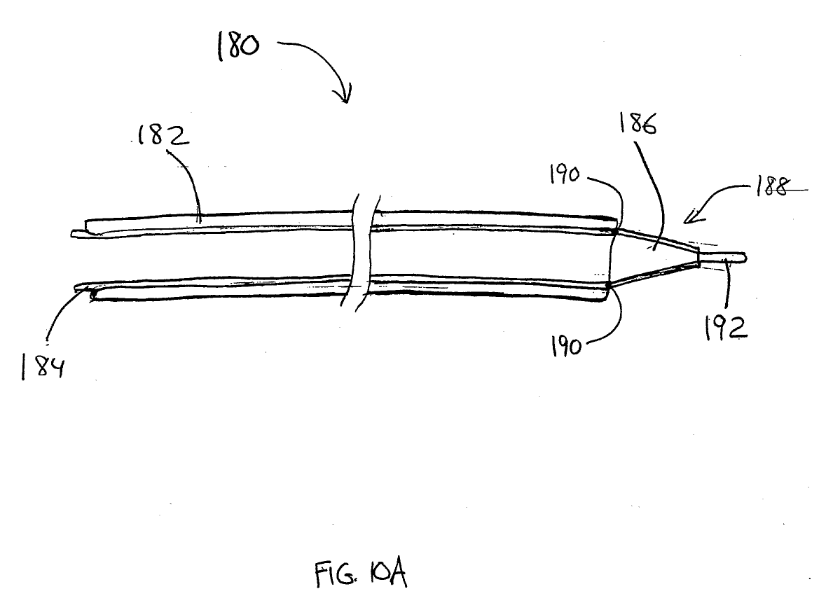

FIGS. 9A to 9C show a partial cut-away view of the colon with the access device 120 with the sealing membrane 128 including the deployable cuff positioned in a stowed state located proximate a target wall segment of the colon via the anus. As shown in FIG. 9B, the elastic cuff covered by (or including) the sealing membrane 128 is then expanded to the deployed state and at least a portion of the sealing membrane is attached to the target wall segment of the colon with pressure and/or an adhesive and/or sealing layer. The adhesive can have one or more of the properties described elsewhere herein.

FIG. 9C shows the sealing membrane 128 attached to the target wall segment of the colon with an incision 160 made in the sealing membrane 148 and the target wall segment with a needle knife, for example (not shown), advanced along the elongate body 124 of the access device 120. In so doing, a sterile pathway is established to the peritoneal cavity. In some embodiments, the device 120 can be air tight to allow pneumoperitoneum and intra abdominal pressure monitoring. The access device can be removed as described above.

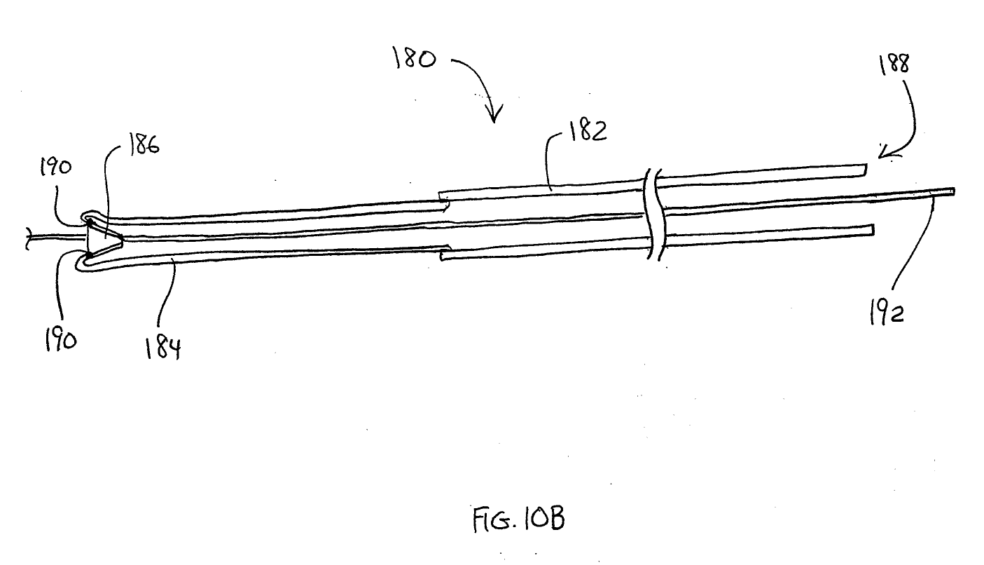

The approaches and devices described herein can be used in combination with a sterile overtube to further protect the oral cavity and esophagus. FIGS. 10A and 10B show one such sterile overtube 180 including an elongate tube 182, an invertible liner 184 releasably attached to an inner surface along the length of the elongate tube 182 and fixedly attached to a savary dilating canula 186, for example, disposed at a distal end 188 of the overtube along a bond 190. A guide wire 192 extends along the elongate tube 182 and beyond the savary dilating canula 186. Once the overtube 182 is positioned in place, the savary dilating canula 186 is withdrawn along the elongate tube 182 with the invertible liner 184 attached thereto as shown in FIG. 10B, and the sterile pathway to the vicinity of the target wall segment is preserved. Thereafter, any endoscopic tools, including the sterile conduit described herein, can be safely passed into the mouth and down the esophagus into the stomach.

### Surgical Procedures and Devices

A variety of procedures can be performed using the new methods, including abdominal exploration, cholecystectomies, spleenectomies, oopherectomies, hysterectomies, tubal ligations, pancreatic surgery, hepatic resections, gastrojejunostomy, thoracic procedures and bariatric procedures such as the Roux-en-Y gastric bypass.

As an alternative to the standard gastric bypass procedures, a Lap-Band can also be applied to the stomach using per-oral or per-anal techniques, e.g., through the new sterile overtube, which is a less invasive bariatric procedure. In this procedure a gastric band device is introduced into the peritoneal cavity through a tiny (1 cm) incision in the wall of the stomach or esophagus and is placed around the upper part of the stomach to form a small (e.g., 15 cc) pouch. The band is designed so that it can be inflated or deflated at any time after the operation with an internal balloon. This helps the patient continually lose weight until they reach their goals.

In some embodiments, the restriction device or band includes an anchoring mechanism, which can be durable and/or reversible. The restrictive device itself is configured to create a small pouch or sleeve of stomach (in the proximal cardia region of the stomach) that is distended with a small meal. In some embodiments, an internal version of the Lap-Band is configured to be adjustable to account for patient adaptation. In other embodiments, the Lap-Band is configured to create a tight gastric sleeve along the lesser curve of the stomach (similar to the vertical banded gastroplasty).

The per-oral and per-anal approaches outlined here can be used individually or simultaneously to perform any intra-abdominal or pelvic surgery. Conventional therapeutic endoscopic devices can be used in combination with the sterile conduit including retractors, needles holders, forceps, scissors, clamps, rongeurs, probes and/or snares, for example. Scopes including gastroscopes, enteroscopes, cystoscopes, and duodenoscopes, for example, can also be used in combination with the approaches described herein.

### EXAMPLES

The following are illustrative examples, and not limiting.

### Example 1

### Endoscopic Partial Hysterectomy in a Porcine Model

Methods: Four-month old female domestic pigs have food withheld for 24 hours and are given antibiotics prior to surgery. After induction of anesthesia, the esophagus is intubated, a sterile access device is advanced along the esophagus, and an antibacterial gastric lavage is perform. The sealing membrane located at the distal end of the access device is positioned on the gastric wall and adhered thereto by endoscopically applied UV light to a UV-curable adhesive disposed on the sealing membrane. Using a needle-knife, a 1 cm incision is made in the sealing membrane and the attached gastric wall. A gastroduodenoscope is pushed through the gastric wall and into the peritoneal cavity. The fallopian tubes, uterus, and ovaries are identified. Forceps are used to pull the uterus and portions of the fallopian tubes through an Olympus® Endo-Loop. The loop is tightly closed and secured. Subsequently, a snare is positioned just distal to the Endo-Loop and cautery is used to resect the uterus and portions of the fallopian tubes. The specimen is removed through the mouth via the gastric incision and is sent for pathology.

The gastric incision is closed using endoscopically placed clips and the access device is separated from the sealing membrane along a frangible region. The sealing membrane remains on the gastric wall until the adhesive biodegrades and the sealing membrane passes harmlessly out of the body along the GI tract.

### Example 2

### Endoscopic Transgastric Oophorectomy and Partial Tubectomy in a Porcine Model

Methods: Female pigs weighing 30 kg are kept without food for one day prior to surgery. Perioperative intravenous antibiotics are administered. After induction of anesthesia, the esophagus is intubated and a sterile access device is placed in the esophagus. Antibacterial gastric lavage is performed and the endoscope is withdrawn. A second sterile dual-channel endoscope is passed through the access device. Endoscopic ultrasound is used to locate a target wall segment suitable for the gastric incision and position the sealing membrane at the target wall segment. The sealing membrane is releasably attached to the target wall segment by activating an adhesive. A transgastric incision is made in the sealing membrane and the target wall segment with a needle-knife.

An endoscope is then introduced into the peritoneal cavity and the Fallopian tubes and ovaries are identified by abdominal exploration. An Olympus Endo-Loop is placed around the left Fallopian tube and a snare is then used to perform partial tubectomy. The ipsilateral ovary is similarly removed and the specimens successfully are retrieved. The contralateral tube and ovary serve as a control. The gastric incision is then closed with endoscopically placed clips and the sealing membrane is removed by lowering the temperature at the target wall segment by delivering saline thereto and deactivating the adhesive on the sealing membrane. The animals are observed overnight and fed a regular diet the following morning.

### Example 3

### Endoscopic Transgastric Abdominal Exploration and Organ Resection: in a Porcine Model

Methods: Female Yorkshire pigs are used for the study. Under general anesthesia, a sterile access device is advanced into the esophagus and a gastroscope is advanced into the access device. Antibacterial lavage is performed and the sealing membrane of the access device is secured to the target segment of the gastric wall with an adhesive applied the sealing membrane. The sealing membrane and gastric wall at the target segment are incised and a sterile dual-channel endoscope is advanced into the peritoneal cavity. Endoscopic abdominal exploration is performed in nine animals and oophorectomy and partial hysterectomy is performed in six animals. The gastric incision is closed with endoclips. The sealing membrane is removed from the target segment by applying force sufficient to overcome the force of the adhesive between the gastric wall and sealing membrane.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of endoscopically accessing a body cavity of a patient, the method comprising:
positioning a scaling membrane through a natural body orifice leading into the gastrointestinal tract of a patient;
advancing the sealing membrane along the gastrointestinal tract toward a target wall segment of the gastrointestinal tract, the target wall segment forming a boundary between the tract and the body cavity;
attaching the scaling membrane to the target wall segment;
forming an incision in the sealing membrane and in the target wall segment; and
introducing an instrument through the incision and into the body cavity.

2. The method of claim 1, wherein the sealing membrane is permanently attachable to the target wall segment.

3-5. (canceled)

6. The method of claim 1, wherein the sealing membrane is releasably attached to the target wall segment.

7. The method of claim 1, further comprising detaching the sealing membrane from the flexible conduit.

8-16. (canceled)

17. An endoscopic access device comprising:
an elongate tube defining an interior passage sized to receive an instrument;
a sealing membrane positioned at the distal end of the tube, the sealing membrane having a stowed state and a deployed state; and
a selectively activatable adhesive disposed on an outer side of the sealing membrane for engagement with a wall segment of a gastrointestinal tract.

18. The device of claim 17, further comprising, at a distal end of the elongate tube, a deployable expandable cuff having a stowed state and a deployed state, wherein
in the stowed state, the cuff is sized for passage through a natural body orifice, and
in the deployed state, the cuff is sized to securely engage a target inner wall segment of the gastrointestinal tract, and
wherein the scaling membrane is an integral portion of the cuff.

19. The device of claim 17 wherein the scaling membrane is sized for passage through a natural body orifice in the stowed state and the sealing membrane is sized to securely engage a target inner wall segment of a gastrointestinal tract in the deployed state.

20. (canceled)

21. The device of claim 19, further comprising an activatable adhesive disposed on an outer side of the sealing membrane for engagement with a wall segment of a gastrointestinal tract.

22. The device of claim 21, wherein the adhesive is activated by a predetermined temperature.

23. (canceled)

24. The device of claim 21, wherein the adhesive is activated by exposure to ultraviolet light.

25. The device of claim 21, wherein the adhesive is activated by exposure to a body fluid.

26. (canceled)

27. The device of claim 21, wherein the adhesive is pressure activatable.

28. The device of claim 21, wherein the adhesive is chemically activatable.

29-34. (canceled)

35. The device of claim 21, wherein the adhesive is configured to degrade over a predetermined time.

36. The device of claim 21, wherein the adhesive is configured to deactivate upon application of at least one of a predetermined temperature, pH level, a predetermined moisture level and a chemical agent.

37. The device of claim 21, further comprising a frangible zone disposed between the elongate tube and the sealing membrane.

38. The device of claim 37, wherein the frangible zone is configured to separate the sealing membrane from the elongate tube upon application of a predetermined force.

39-42. (canceled)

43. A method of endoscopically accessing a body cavity of a patient, the method comprising:
positioning the access device of claim 17 through a natural body orifice leading into the gastrointestinal tract of a patient;
advancing the sealing membrane along the gastrointestinal tract toward a target wall segment of the gastrointestinal tract;
activating an adhesive disposed on an outer side of the scaling membrane;
attaching the sealing membrane to the target wall segment; and
forming an incision in the sealing membrane and in the target wall segment to provide access to the body cavity.

44. A method of endoscopically accessing a body cavity of a patient, the method comprising:
positioning the access device of claim 18 through a natural body orifice and along the gastrointestinal tract of a patient;
advancing the cuff along the gastrointestinal tract until proximate a target wall segment of the gastrointestinal tract;
expanding the cuff from a stowed to a deployed state to secure the cuff to the target wall segment; and
forming an incision in the cuff and target wall segment secured thereto to provide access to the body cavity.

45-48. (canceled)

49. The device of claim 17, further comprising a pressure controller in communication with the sealing membrane.
